(19) United States
(12) Patent Application Publication
(10) Pub. No.: US 2020/0048675 A1
(43) Pub. Date: Feb. 13, 2020

(54) PRODUCTION METHOD FOR SUBSTANCE USING ATP

(71) Applicant: KANEKA CORPORATION, Osaka (JP)

(72) Inventors: Misato Matsui, Hyogo (JP); Noriyuki Ito, Hyogo (JP); Yoshihiko Yasohara, Hyogo (JP)

(73) Assignee: KANEKA CORPORATION, Osaka (JP)

(21) Appl. No.: 16/668,197

(22) Filed: Oct. 30, 2019

Related U.S. Application Data
(63) Continuation of application No. PCT/JP2018/016175, filed on Apr. 19, 2018.

(30) Foreign Application Priority Data
May 1, 2017 (JP) 2017-091451

(51) Int. Cl. C12P 21/02; C12N 9/12; C12N 9/00

(52) U.S. Cl. CPC C12P 21/02; C12N 9/1229; C12Y 603/02003; C12N 9/93; C12Y 603/02002; C12Y 207/04001

(57) ABSTRACT

A method of producing a substance includes synthesizing a molecule at least by mixing substrates, a synthase, adenosine triphosphate (ATP), a polyphosphate kinase 2, and a polyphosphoric acid mixture. The polyphosphoric acid mixture includes 50% or more of polyphosphoric acid with a degree of polymerization of not less than 15. Adenosine diphosphate (ADP) is generated from the ATP during the synthesis. The synthesis is coupled with an ATP regeneration reaction in which the ATP is regenerated by the polyphosphate kinase 2 from the ADP and the polyphosphoric acid.

### TECHNICAL FIELD

One or more embodiments of the present invention relate to a novel method of producing a substance using adenosine triphosphate (ATP).

### BACKGROUND ART

Glutathione is a peptide composed of the following three amino acids: L-cysteine, L-glutamic acid, and glycine. Glutathione can be found not only in human bodies but also in many other living bodies such as other animals, plants, and microorganisms. Furthermore, glutathione has the functions of eliminating reactive oxygen, detoxification, amino acid metabolism, and the like, and is a compound important to living bodies.

Glutathione in vivo is in the form of (i) reduced glutathione (hereinafter may be referred to as “GSH”), in which the thiol group of L-cysteine residue is in a reduced form “—SH” or (ii) oxidized glutathione (hereinafter may be referred to as “GSSG”), in which the thiol groups of L-cysteine residues of two glutathione molecules are oxidized to form a disulfide bond between the two glutathione molecules.

Examples of a known method of producing glutathione include an enzymatic production in which bodies of Escherichia coli and/or Saccharomyces cerevisiae, which have been recombined to produce γ-glutamylcysteine synthase and/or glutathione synthase, are used as enzyme sources in the presence of L-glutamic acid, L-cysteine, glycine, a surfactant, an organic solvent and/or the like (Patent Literatures 1 and 2). Furthermore, the applicant has recently disclosed a method of producing oxidized glutathione, the method including the steps of: producing oxidized γ-glutamylcysteine from L-glutamic acid and L-cystine; and then producing oxidized glutathione from the oxidized γ-glutamylcysteine and glycine (Patent Literature 3).

Examples of a known enzyme involved in glutathione synthesis include: γ-glutamylcysteine synthase (hereinafter may be referred to as “GSHI”) which combines L-glutamic acid and L-cysteine to form γ-glutamylcysteine; and glutathione synthase (hereinafter may be referred to as “GSHII”) which combines γ-glutamylcysteine and glycine to form reduced glutathione. The GSHI and GSHII are known to be capable of also using L-cystine and oxidized γ-glutamylcysteine as substrates, respectively. In a case where the GSHI and GSHII use L-cystine and oxidized γ-glutamylcysteine as substrates, respectively, their enzymatic reactions result in synthesis of oxidized γ-glutamylcysteine and oxidized glutathione, respectively, as reaction products (Patent Literature 3). Furthermore, bifunctional glutathione synthase (hereinafter may be referred to as “GSHF”) which has both functions of the GSHI and GSHII is also known (Patent Literature 3).

[Patent Literature 1]

Japanese Patent Application Publication, Tokukaisho, No. 60-27396

[Patent Literature 2]

Japanese Patent Application Publication, Tokukaisho, No. 60-27397

[Patent Literature 3]

PCT International Publication No. WO 2016/002884

Incidentally, the GSHI, GSHII, GSHF, and the like consume adenosine triphosphate (hereinafter may be referred to as “ATP”) as an energy source for their activity. Therefore, in order to maintain the reaction of glutathione production, it is necessary to externally supply ATP or it is necessary to reconvert adenosine diphosphate (hereinafter may be referred to as “ADP”), which is a product of the consumption of ATP, into ATP.

External supply of ATP is very costly; therefore, an ATP-regenerating system, in which ADP is reconverted into ATP, has been considered for application.

A known enzyme that converts ADP to ATP in the ATP-regenerating system is a polyphosphate kinase 2. This enzyme has the function of converting ADP into ATP using metaphosphoric acid or the like as a substrate.

However, a production method in which the ATP-regenerating system is included as part of the production of a substance, for example, a method of producing oxidized glutathione, has been required to have an improved ATP-regenerating system in order to achieve a higher rate of conversion from a source material into a final product (e.g., oxidized glutathione).

### SUMMARY

One or more embodiments of the present invention provide a novel method of producing a substance using ATP.

The inventors for the first time found that, by using, as a substrate for a polyphosphate kinase 2, a mixture that contains polyphosphoric acid molecules with a high degree of polymerization, it is possible to achieve a high rate of conversion to oxidized glutathione. On the basis of this finding, the inventors accomplished one or more embodiments of the present invention.

Specifically, one or more embodiments of the present invention relate to a method of producing a substance using ATP, wherein: ADP is generated from ATP during the method; the method is coupled with an ATP regeneration reaction in which a polyphosphate kinase 2 and polyphosphoric acid are allowed to react with the ADP to regenerate ATP; and the ATP used in the method includes the ATP regenerated by the ATP regeneration reaction, the method including using, as a substrate for the polyphosphate kinase 2, a polyphosphoric acid mixture that contains polyphosphoric acid molecules with a degree of polymerization of not less than 15 in an amount of not less than 48%.

According to one or more embodiments of the present invention, it is possible to produce a substance using ATP with a high conversion rate at low cost.

### BRIEF DESCRIPTION OF DRAWINGS

FIG. 1 is a chart showing the results of analysis of a polyphosphoric acid mixture in terms of the degree of polymerization.

FIG. 2 is a chart that shows a comparison, in terms of changes in degree of polymerization, between polyphosphoric acid mixtures which had been left to stand for different periods of time after their preparations.

FIG. 3 shows charts showing how consumption of a polyphosphoric acid mixture changes during production of oxidized glutathione.

### DETAILED DESCRIPTION OF EMBODIMENTS

The following description will discuss, in detail, one or more embodiments of the present invention. Note that all academic and patent literatures listed herein are incorporated herein by reference.

In this specification, the term “gene” is used interchangeably with the term “polynucleotide”, “nucleic acid” or “nucleic acid molecule”, and is intended to mean a polymer of nucleotides. A gene can exist in the form of DNA (e.g., cDNA or genomic DNA) or RNA (e.g., mRNA). DNA or RNA may be double-stranded or single stranded. Single-stranded DNA or RNA may be a coding strand (sense strand) or may be a non-coding strand (antisense strand). A gene may be chemically synthesized, and may have codon usage modified so that the expression of a protein that the gene codes for improves. Codons which code for the same amino acid may be replaced with each other.

The term “protein” is used interchangeably with the term “peptide” or “polypeptide”. In this specification, bases and amino acids are indicated by single letter codes or three letter codes of IUPAC standards and IUB standards.

[Method of Producing Substance]

One or more embodiments of the present invention provide a method of producing a substance using ATP, wherein: ADP is generated from ATP during the method; the method is coupled with an ATP regeneration reaction in which a polyphosphate kinase 2 and polyphosphoric acid are allowed to react with the ADP to regenerate ATP; and the ATP used in the method includes the ATP regenerated by the ATP regeneration reaction, the method including using, as a substrate for the polyphosphate kinase 2, a polyphosphoric acid mixture that contains polyphosphoric acid molecules with a degree of polymerization of not less than 15 in an amount of not less than 48%.

One or more embodiments of the present invention were accomplished based on the following finding. The inventors for the first time found that, by arranging a method of producing a substance using ATP such that ATP is regenerated using, as a substrate for a polyphosphate kinase 2, a polyphosphoric acid mixture containing a certain amount or more of polyphosphoric acid molecules with a specific degree of polymerization (particularly, polyphosphoric acid molecules with a high degree of polymerization), it is possible to produce a substance using the ATP with a high conversion rate. As such, one or more embodiments of the present invention use a polyphosphoric acid mixture that contains a certain amount or more of polyphosphoric acid molecules with a high degree of polymerization in the ATP regeneration reaction, and thereby makes it possible to produce a substance with a high conversion rate at low cost.

The following description will discuss one or more embodiments of the present invention in detail.

<1. Polyphosphoric Acid Mixture>

In one or more embodiments of the present invention, it is preferable that the following are used: a polyphosphate kinase 2; and a polyphosphoric acid mixture that serves as a substrate for the polyphosphate kinase 2 and that contains polyphosphoric acid molecules with a degree of polymerization of not less than 15 in an amount of not less than 48%. In one or more embodiments of the present invention, use of such a polyphosphoric acid mixture that contains a certain amount or more of polyphosphoric acid molecules with a high degree of polymerization makes it possible to produce a substance with a high conversion rate at low cost.

In this specification, the term “polyphosphoric acid” is intended to mean a polymer obtained by polymerization of phosphoric acid units. For example, a polyphosphoric acid is a compound represented by Formula 1 below.

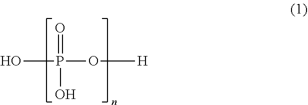

In this specification, the term “metaphosphoric acid” is intended to mean a compound that contains (i) a chain polymer structure composed of phosphoric acid units and (ii) a ring structure. A “metaphosphoric acid” is, for example, a compound that contains a compound represented by Formula (1) (corresponding to “chain polymer structure composed of phosphoric acid units”) and a compound represented by Formula 2 below (corresponding to “ring structure”).

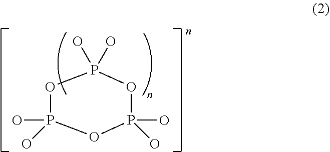

In this specification, the term “polyphosphoric acid mixture” is intended to mean a mixture that contains one of the “polyphosphoric acid” and “metaphosphoric acid” or that contains both of the “polyphosphoric acid” and “metaphosphoric acid”. The proportion of the “polyphosphoric acid” and/or “metaphosphoric acid” in the “polyphosphoric acid mixture” is not particularly limited, provided that the effects according to one or more embodiments of the present invention are achieved.

Note that, because the “polyphosphoric acid” and “metaphosphoric acid” can be present in a mixed manner, it is difficult to strictly separate them from each other. Therefore, in this specification, the terms “polyphosphoric acid” and “metaphosphoric acid” are not distinguished precisely. The term “polyphosphoric acid” herein means “polyphosphoric acid” that contains “metaphosphoric acid”, and the term “metaphosphoric acid” herein means “metaphosphoric acid” that contains “polyphosphoric acid”.

In one or more embodiments of the present invention, the polyphosphoric acid mixture contains polyphosphoric acid molecules with a degree of polymerization of not less than 15 in an amount of not less than 48%, preferably contains polyphosphoric acid molecules with a degree of polymerization of not less than 15 in an amount of not less than 50%.

In one or more embodiments of the present invention, the polyphosphoric acid mixture contains polyphosphoric acid molecules with a degree of polymerization of not less than 20 in an amount of not less than 31%, preferably contains polyphosphoric acid molecules with a degree of polymerization of not less than 20 in an amount of not less than 32%.

In one or more embodiments of the present invention, the polyphosphoric acid mixture contains polyphosphoric acid molecules with a degree of polymerization of not less than 36 in an amount of not less than 4%, preferably contains polyphosphoric acid molecules with a degree of polymerization of not less than 36 in an amount of not less than 5%.

In one or more embodiments of the present invention, the polyphosphoric acid mixture contains polyphosphoric acid molecules with a degree of polymerization of not less than 43 in an amount of not less than 2%. In one or more embodiments of the present invention, the polyphosphoric acid mixture contains polyphosphoric acid molecules with a degree of polymerization of not less than 50 in an amount of not less than 2/a %.

The degree of polymerization of polyphosphoric acid molecules in one or more embodiments of the present invention is determined by a method that will be described later in Examples. Furthermore, examples of such a polyphosphoric acid mixture that contains a certain amount or more of polyphosphoric acid molecules with a high degree of polymerization will be provided later in Examples (see Examples 1, 4, and the like).

<2. Polyphosphate Kinase 2 (PPK2)>

One or more embodiments of the present invention provide a method of producing a substance, in which the polyphosphate kinase 2 is at least one selected from the group consisting of: polyphosphate kinase 2 derived from Pseudomonas aeruginosa (hereinafter may be referred to as “PNDK”); polyphosphate kinase 2 derived from Synechococcus sp. PCC6312 (hereinafter may be referred to as “Sy PPK2”; polyphosphate kinase 2 derived from Corynebacterium efficiens (hereinafter may be referred to as “CE PPK2”); polyphosphate kinase 2 derived from Kineococcus radiotolerans (hereinafter may be referred to as “KR PPK2”); polyphosphate kinase 2 derived from Pannonibacter indicus (hereinafter may be referred to as “PI PPK2”); polyphosphate kinase 2 derived from Deinococcus radiodurans K1 (hereinafter may be referred to as “DR PPK2”); polyphosphate kinase 2 derived from Gulbenkiania indica (hereinafter may be referred to as “GI PPK2”); polyphosphate kinase 2 derived from Arthrobactor aurescens TC1 (hereinafter may be referred to as “AA PPK2”); polyphosphate kinase 2 derived from Thiobacillus denitrificans ATCC25259 (hereinafter may be referred to as TD PPK2”); and polyphosphate kinase 2 derived from Pseudomonas fluorescens (hereinafter may be referred to as “PF PPK2”).

Polyphosphate kinases (hereinafter may be referred to as “PPK”) are classified into two types of enzyme for a reversible reaction: polyphosphate kinase 1 (hereinafter may be referred to as “PPK1”): and polyphosphate kinase 2 (hereinafter may be referred to as “PPK2”). It is known that the PPK1s are predominantly involved in a reaction that degrades ATP into ADP and polyphosphoric acid (hereinafter may be referred to as “PolyP”) and that the PPK2s are predominantly involved in a reaction that combines ADP and PolyP to form ATP.

The PPK2s are further classified into three classes in terms of the reactions they catalyze. Class I PPK2 catalyzes a reaction that combines ADP and PolyP to form ATP, and examples thereof include PNDK. Class II PPK2 catalyzes a reaction that combines adenosine monophosphate (hereinafter may be referred to as “AMP”) and PolyP to form ATP, and examples thereof include polyphosphoric-acid-dependent AMP transferase (PAP). Class III PPK2 is a bifunctional enzyme that catalyzes the two reactions of the above Class I and Class II, and examples thereof include PPK2 derived from Meiothermus ruber.

The inventors have studied hard in order to search for a novel PPK2, and succeeded in identifying eight types of novel PPK2 which are classified into Class I or Class III and which have the activity of combining ADP and PolyP to thereby convert ADP into ATP. This makes it possible to catalyze the ATP regeneration reaction by use of a PPK2 selected appropriately from not only conventionally-known PPK2s and DR PPK2 but also these eight types of PPK2.

Needless to say, in one or more embodiments of the present invention, a conventionally-known PPK2 can be employed as the polyphosphate kinase 2.

The following description discusses the above PPK2s (i.e., PNDK, DR PPK2, and eight types of novel PPK2) in detail.

The PNDK is polyphosphate kinase 2 derived from Pseudomonas aeruginosa, and is composed of a total of 357 amino acid residues (SEQ ID NO:1).

The Sy PPK2 is polyphosphate kinase 2 derived from Synechococcus sp. PCC6312, and is composed of a total of 296 amino acid residues (SEQ ID NO:2).

The CE PPK2 is polyphosphate kinase 2 derived from Corynebacterium efficiens, and is composed of a total of 351 amino acid residues (SEQ ID NO:3).

The KR PPK2 is polyphosphate kinase 2 derived from Kineococcus radiotolerans, and is composed of a total of 296 amino acid residues (SEQ ID NO:4).

The PI PPK2 is polyphosphate kinase 2 derived from Pannonibacter indicus, and is composed of a total of 367 amino acid residues (SEQ ID NO:5).

The DR PPK2 is polyphosphate kinase 2 derived from Deinococcus radiodurans K1, and is composed of a total of 266 amino acid residues (SEQ ID NO:6).

The GI PPK2 is polyphosphate kinase 2 derived from Gulbenkiania indica, and is composed of a total of 350 amino acid residues (SEQ ID NO:7).

The AA PPK2 is polyphosphate kinase 2 derived from Arthrobactor aurescens TC1, and is composed of a total of 314 amino acid residues (SEQ ID NO:8).

The TD PPK2 is polyphosphate kinase 2 derived from Thiobacillus denitrificans ATCC25259, and is composed of a total of 269 amino acid residues (SEQ ID NO:9).

The PF PPK2 is polyphosphate kinase 2 derived from Pseudomonas fluorescens, and is composed of a total of 362 amino acid residues (SEQ ID NO:10).

The following are base sequences which code for the above ten types of PPK2 and which are codon-optimized for expression in E. coli: PNDK (SEQ ID NO:11); Sy PPK2 (SEQ ID NO:12); CE PPK2 (SEQ ID NO:13); KR PPK2 (SEQ ID NO:14); PI PPK2 (SEQ ID NO:15); DR PPK2 (SEQ ID NO:16); GI PPK2 (SEQ ID NO:17); AA PPK2 (SEQ ID NO:18); TD PPK2 (SEQ ID NO:19); and PF PPK2 (SEQ ID NO:20).

The PNDK has an optimum temperature of 37° C. (Motomura et al., Applied and Environmental Microbiology, volume 80, number 8, 2602-2608, 2014). Therefore, use of the PNDK makes it possible to carry out a reaction at relatively low temperature (that is, under moderate conditions). In view of this, the PNDK is therefore preferred as the PPK2 in one or more embodiments of the present invention.

Furthermore, as described earlier, the PNDK is PPK2 derived from Pseudomonas aeruginosa. Therefore, provided that a PPK2 is derived from a microbial species classified in the Pseudomonas genus, this PPK2 can have similar advantages to the foregoing advantages of the PNDK. Thus, a PPK2 derived from a microbial species classified in the Pseudomonas genus is preferred as the PPK2 in one or more embodiments of the present invention.

Other examples of a microbial species classified in the Pseudomonas genus, other than the foregoing Pseudomonas aeruginosa and Pseudomonas fluorescens, include the following species: Pseudomonas oxalaticus, Pseudomonas stuzeri, Pseudomonas chloraphis, Pseudomonas riboflavina, Pseudomonas fragi, Pseudomonas mendocina, Pseudomonas sp. K-9, Pseudomonas diminuta, Pseudomonas vesicularis, Pseudomonas caryophylli, Pseudomonas cepacian, Pseudomonas antimicrobica, Pseudomonas plantarii, Pseudomonas marina, Pseudomonas testosterone, Pseudomonas lanceolate, Pseudomonas acidovorans, Pseudomonas rubrisubalbicans, Pseudomonas flava, Pseudomonas palleronii, Pseudomonas pseudoflava, Pseudomonas taeniospiralis, Pseudomonas nautica, Pseudomonas iners, Pseudomonas mesophilica, Pseudomonas radiora, Pseudomonas rhodos, Pseudomonas doudoroffii, Pelomonas saccharophila, Pseudomonas abietaniphila, Pseudomonas alcaligenes, Pseudomonas alcaliphila, Pseudomonas auricularis, Pseudomonas azotoformans, Pseudomonas balearica, Pseudomonas chlororaphis subsp. aureofaciens, Pseudomonas chlororaphis subsp. chlororaphis, Pseudomonas citronellolis, Pseudomonas cremoricolorata, Pseudomonas flavescens, Pseudomonas fragi, Pseudomonas fulva, Pseudomonas gessardii, Pseudomonas indica, Pseudomonas japonica, Pseudomonas jianii, Pseudomonas jinjuensis, Pseudomonas luteola, Pseudomonas mandelii, Pseudomonas mendocina, Pseudomonas migulae, Pseudomonas monteilii, Pseudomonas mucidolens, Pseudomonas nitroreducens, Pseudomonas nitroreducens subsp. thermotolerans, Pseudomonas oleovorans, Pseudomonas oryzihabitans, Pseudomonas parafulva, Pseudomonas pavonaceae, Pseudomonas pertucinogena, Pseudomonas plecoglossicida, Pseudomonas pseudoalcaligenes, Pseudomonas reptilivora, Pseudomonas resinovorans, Pseudomonas sp., Pseudomonas straminea, Pseudomonas striafaciens, Pseudomonas syncyanea, Pseudomonas synxantha, Pseudomonas syringae, Pseudomonas taetrolens, Pseudomonas tolaasii, Pseudomonas toyotomiensis, Pseudomonas pickettii, Pseudomonas echinoides, Pseudomonas paucimobilis, Pseudomonas maltophilia, and Pseudomonas butanovora.

In one or more embodiments of the present invention, the PPK2 may be in the form of (i) a live cell of an organism having the PPK2 activity, (ii) a dead but undamaged cell of an organism having the PPK2 activity, or (iii) a protein that has been isolated from the cell and purified. The degree of purification of the protein that has the PPK2 activity here is not limited to a particular degree, and the purification may be partial purification. The PPK2 may be a freeze-dried or acetone-dried body that has the PPK2 activity, may be the body which has been triturated, or may be a polypeptide itself fixed or a body fixed as-is.

In one or more embodiments, it is preferable not to use live cells having the PPK2 activity. In one or more embodiments, it is more preferable to use neither live cells having the PPK2 activity nor undamaged dead cells.

In one or more embodiments of the present invention, each of the foregoing ten types of PPK2 may be a protein which (i) has the same amino acid sequence as shown in a corresponding one of SEQ ID NOs: 1 to 10 except that one to several amino acid residues are substituted, deleted inserted and/or added and (ii) has the PPK2 activity (such proteins are hereinafter referred to as proteins of case (a)).

The specific sequence of each protein of case (a) is not limited, provided that the sequence constitutes a protein which (i) is a mutant, a derivative, a variant, an allele, a homologue, an orthologue, a partial peptide, a fusion protein with some other protein/peptide, or the like, each of which is functionally equivalent to a corresponding one of the proteins having the amino acid sequences shown in SEQ ID NOs: 1 to 10 and (ii) has the PPK2 activity. The number of amino acids that may be deleted, substituted or added is not limited, provided that the foregoing function is not impaired, and is intended to mean the number of amino acids that can be deleted, substituted or added by a known insertion method such as site-directed mutagenesis. In one or more embodiments, the number of such amino acids is preferably five or less, more preferably three or less (e.g., three amino acids, two amino acids, or one amino acid). In this specification, the term “mutation” mainly refers to a mutation artificially introduced by, for example, site-directed mutagenesis; however, the term “mutation” may refer to an equivalent naturally-occurring mutation.

In one or more embodiments, in a case where an amino acid residue is substituted, it is preferable that the amino acid residue is substituted with another amino acid whose side chain has the same property. Examples of properties of amino acid side chains include: hydrophobic amino acids (A, I, L, M, F, P, W, Y, V); hydrophilic amino acids (R, D, N, C, E, Q, G, H, K, S, T); amino acids with aliphatic side chain (G, A, V, L, I, P); amino acids with hydroxyl-containing side chain (S, T, Y); amino acids with sulfur-atom-containing side chain (C, M); amino acids with carboxylic-acid-and-amide-containing side chain (D, N, E, Q); amino acids with base-containing side chain (R, K, H); and amino acids with aromatic-containing side chain (H, F, Y, W) (the letters provided in parentheses are each a single letter code of an amino acid). It is known that a polypeptide having a certain amino acid sequence maintains its biological activity even if one to several amino acid residues in the amino acid sequence are deleted, added and/or substituted by some other amino acid and thereby the amino acid sequence is modified. In one or more embodiments, it is preferable that a mutated amino acid residue and the original amino acid residue have as many common properties as possible.

In this specification, the phrase “functionally equivalent” is intended to mean that a certain protein has a biological function and/or a biochemical function equivalent to (identical to and/or similar to) a target protein. Biological properties can include specificity with regard to expression site, expression level, and the like. Whether or not the protein having a mutation(s) introduced therein has a desired function can be determined by (i) obtaining a transformant in which a gene coding for that protein is introduced and expressed and (ii) checking whether this transformant can generate ATP from ADP and PolyP.

In one or more embodiments of the present invention, each of the foregoing ten types of PPK2 may be a protein which (i) has a sequence homology of not less than 80% with the amino acid sequence shown in a corresponding one of SEQ ID NOs:1 to 10 and (ii) has the PPK2 activity (such proteins are hereinafter referred to as proteins of case (b)).

The specific sequence of each protein of case (b) is not limited, provided that the sequence constitutes a protein which (i) is a mutant, a derivative, a variant, an allele, a homologue, an orthologue, a partial peptide, a fusion protein with some other protein/peptide, or the like, each of which is functionally equivalent to a corresponding one of the proteins having the amino acid sequences shown in SEQ ID NOs: 1 to 10 and (ii) has the PPK2 activity.

In one or more embodiments, the phrase “an amino acid sequence has a homology with another amino acid sequence” means that at least 80%, more preferably not less than 90%, even more preferably not less than 95% (for example, not less than 95%, not less than 96%, not less than 97%, not less than 98%, not less than 99%) of the entire amino acid sequence (or an entire region that is necessary for functional expression) is identical to that of the another amino acid sequence. The homology of an amino acid sequence can be determined with use of a BLASTN program (nucleic acid level) or a BLASTX program (amino acid level) (Altschul et al. J. Mol. Biol., 215: 403-410, 1990). These programs are based on the algorithm BLAST by Karlin and Altschul (Proc. Natl. Acad. Sci. USA, 87:2264-2268, 1990, Proc. Natl. Acad. Sci. USA, 90: 5873-5877, 1993). In a case where a base sequence is analyzed by BLASTN, the parameters employed here are, for example, score=100 and wordlength=12. In a case where an amino acid sequence is analyzed by BLASTX, parameters employed here are, for example, score=50 and wordlength=3. In a case where an amino acid sequence is analyzed with use of Gapped BLAST program, such an analysis can be carried out as disclosed in Altschul et al. (Nucleic Acids Res. 25: 3389-3402, 1997). In a case of using the BLAST program and the Gapped BLAST program, default parameters of these programs are used. Specific methods of these analyses are known to those skilled in the art. For a comparative base sequence or amino acid sequence to be aligned optimally, addition or deletion (for example, introduction of a gap) may be permitted.

In one or more embodiments, the term “homology” is intended to mean the proportion of amino acid residues that have a similar property to those of a comparative sequence (e.g., homology, positive); however, the “homology” is preferably the proportion of amino acid residues that are identical to those of the comparative sequence. In one or more embodiments, the “homology” is preferably “identity”. Note that the properties of amino acid sequences have already been discussed earlier.

In one or more embodiments of the present invention, each of the foregoing ten types of PPK2 may be a protein that is encoded by a gene having a base sequence shown in a corresponding one of SEQ ID NOs:11 to 20 (such proteins are hereinafter referred to proteins of case (c)).

With regard to the proteins of case (c), SEQ ID NOs: 11 to 20 show base sequences (open reading frames: ORFs) of genes coding for proteins having amino acid sequences shown in SEQ ID NOs: 1 to 10, respectively.

In one or more embodiments of the present invention, in a case where the foregoing ten types of PPK2 are the proteins of case (c), the proteins may be those encoded by genes having modified versions of the respective base sequences of SEQ ID NOs: 11 to 20 which have been modified for, for example, an improvement in expression in a host cell. Each of the proteins of case (c) may have its N-terminus cut off or may have been cleaved at some other position, for the purpose of improvement in expression of the PPK2 in a host cell. Codons may be optimized for the same purpose.

One example of modification of a base sequence is, as shown in Example 3 (described later), to cut off 81 amino acids at the N terminus of wild-type PNDK and substitute alanine at position 82 with methionine which is an initiation codon. Other examples include, as shown in Example 2 (described later): (i) removing amino acids at positions 1 to 85 at the N terminus of an amino acid sequence from native PF PPK2 and introducing a P86M mutation; and (ii) removing amino acids at positions 1 to 86 at the N terminus and introducing a G87M mutation.

The foregoing genes/proteins may be obtained by a usually-used polynucleotide modification method. Specifically, substitution, deletion, insertion and/or addition of a specific base(s) in a polynucleotide that carries genetic information of a protein make it possible to prepare a polynucleotide that carries genetic information of a desired recombinant protein. A specific method of converting a base of a polynucleotide is, for example, use of a commercially-available kit (KOD-Plus Site-Directed Mutagenesis Kit [TOYOBO], Transformer Site-Directed Mutagenesis Kit [Clontech], QuickChange Site Directed Mutagenesis Kit [Stratagene] or the like) or use of a polymerase chain reaction (PCR). These methods are known to those skilled in the art.

Each of the foregoing genes may consist only of a polynucleotide that codes for a corresponding protein, but may have some other base sequence added thereto. The base sequence added is not particularly limited, and examples thereof include: base sequences coding for a label (e.g., histidine tag, Myc tag, FLAG tag, or the like); base sequences coding for a fusion protein (e.g., streptavidin, cytochrome, GST, GFP, MBP or the like): base sequences coding for a promoter sequence; and base sequences coding for a signal sequence (e.g., endoplasmic reticulum translocation signal sequence, secretion sequence, or the like). The site at which any of such base sequences is added is not particularly limited. The base sequence may be added to, for example, a site corresponding to the N terminus or C terminus of a translated protein.

<3. PPK2 Gene>

One or more embodiments of the present invention provide a PPK2 gene coding for any of the foregoing proteins.

The PPK2 gene may either be a nucleotide composed of a native sequence or a nucleotide composed of an artificially modified sequence. In one or more embodiments, the PPK2 gene is preferably a nucleotide composed of a sequence which has been subjected to codon optimization for expression in a host cell (for example, E. coli).

<4. Vector>

One or more embodiments of the present invention provide a vector that contains a gene discussed in the <3. PPK2 gene> section. Examples of the vector not only include expression vectors for expressing the gene in a host cell in order to prepare a transformant but also include those which are for use in production of a recombinant protein.

A base vector serving as a base for the above vector can be any of various kinds of commonly-used vectors. Examples include plasmids, phages and cosmids, from which a base vector can be selected appropriately according to a cell to which it is introduced and how it is introduced. That is, the vector is not limited to a specific kind, and any vector that can be expressed in a host cell can be selected as appropriate. An appropriate promoter sequence for unfailingly expressing the gene may be selected according to the type of host cell, and this promoter sequence and the foregoing gene may be incorporated into a plasmid or the like to obtain a vector. Such a vector may be used as the expression vector. Examples of the expression vector that can be employed include: phage vectors, plasmid vectors, viral vectors, retroviral vectors, chromosome vectors, episome vectors, and virus-derived vectors (for example, bacterial plasmids, bacteriophages, yeast episomes, yeast chromosomal elements and viruses [for example, baculovirus, papovavirus, saccinia virus, adenovirus, avipoxvirus, pseudorabies virus, herpesvirus, lentivirus and retrovirus]); and vectors derived from combinations thereof (for example, cosmids and phagemids).

Examples of a vector suitable for use in bacteria include: pQE30, pQE60, pQE70, pQE80 and pQE9 (available from Qiagen); pTipQC1 (available from Qiagen or Hokkaido System Science Co., Ltd.), pTipRT2 (available from Hokkaido System Science Co., Ltd.); pBS vector, Phagescript vector, Bluescript vector, pNH8A, pNH16A, pNH18A and pNH46A (available from Stratagene); ptrc99a, pKK223-3, pKK233-3, pDR540 and pRIT5 (available from Addgene); pRSF (available from MERCK); and pAC (available from NIPPON GENE CO., LTD.). In particular, examples of a vector suitable for use in a case of E. coli include pUCN18 (which can be prepared by modifying pUC18 available from Takara Bio Inc.), pSTV28 (available from Takara Bio Inc.), and pUCNT (PCT International Publication No. WO 94/03613).

In one or more embodiments, the insertion of the foregoing gene is preferably such that the gene is operatively linked to an appropriate promoter. The other appropriate promoters can be those known to those skilled in the art, and are not particularly limited. Examples of the promoter include: lacUV5 promoter, trp promoter, trc promoter, tac promoter, lpp promoter, tufB promoter, recA promoter, pL promoter, lacI promoter, lacZ promoter, T3 promoter, T5 promoter, T7 promoter, gap promoter, OmpA promoter, and SV40 early promoter and late promoter; and retrovirus LTR promoter.

In one or more embodiments, the vector preferably further contains sites for transcription initiation and transcription termination and contains, within a transcribed region, a site for ribosome binding for translation. A region coding for a mature transcript expressed by a vector construct contains a transcription initiation AUG at the start of a to-be-translated polypeptide and contains a stop codon positioned appropriately at the end.

A host into which a vector is introduced is not particularly limited. Any of various kinds of cells can be used suitably. In one or more embodiments, typical examples of an appropriate host include bacteria, yeast, filamentous fungi, plant cells, and animal cells. E. coli is particularly preferred. An appropriate culture medium and conditions for the above host cell can be any of those known in this technical field.

A method of introducing the foregoing vector into a host cell, that is, a method of transformation, is not particularly limited. Suitable examples include conventionally known methods such as electroporation, calcium phosphate transfection, liposome transfection, DEAE-dextran transfection, microinjection, cationic lipid-mediated transfection, electroporation, transduction, and infection. Such methods are stated in many standard laboratory manuals such as Basic Methods In Molecular Biology (1986) by Davis et al.

<5. Transformant>

One or more embodiments of the present invention provide a transformant that contains a gene discussed in the <3. Gene> section or a recombinant vector discussed in the <4. Vector> section. As used herein, the phrase “contains a gene or a vector” is intended to mean that the gene or vector has been introduced in a target cell (host cell) by a known genetic engineering procedure (gene manipulation technique) such that the gene can be expressed. The meaning of the term “transformant” includes not only cells, tissues, and organs but also living individuals.

The transformant can be prepared (produced) by, for example, transforming an organism with the foregoing vector. The organism to be transformed is not particularly limited, and can be, for example, any of various kinds of organism exemplary listed earlier for the host cell.

A host cell for use in one or more embodiments of the present invention is not particularly limited, provided that the cell allows the expression of an introduced gene or of a protein encoded by a gene contained in a vector. Examples of a microorganism available for use as a host cell include: bacteria such as those belonging to the genus Escherichia, those belonging to the genus Bacillus, those belonging to the genus Pseudomonas, those belonging to the genus Serratia, those belonging to the genus Brevibacterium, those belonging to the genus Corynebacterium, those belonging to the genus Streptococcus, and those belonging to the genus Lactobacillus; actinomycetes such as those belonging to the genus Rhodococcus and those belonging to the genus Streptomyces; yeast such as those belonging to the genus Saccharomyces, those belonging to the genus Kluyveromyces, those belonging to the genus Schizosaccharomyces, those belonging to the genus Zygosaccharomyces, those belonging to the genus Yarrowia, those belonging to the genus Trichosporon, those belonging to the genus Rhodosporidium, those belonging to the genus Pichia, and those belonging to the genus Candida; and fungi such as those belonging to the genus Neurospora, those belonging to the genus Aspergillus, those belonging to the genus Cephalosporium, and those belonging to the genus Trichoderma. Not only microorganisms but also plant cells, animal cells and the like can be used as a host cell. In one or more embodiments, a bacterium is preferred in view of introduction and expression efficiency, and E. coli is particularly preferred.

[Production of Substance Using ATP]

In one or more embodiments of the present invention, production of a substance using ATP is not particularly limited, provided that the method is to produce a substance at the expense of energy derived from ATP. Examples of production of a substance using ATP include: production of oxidized glutathione, production of reduced glutathione, production of S-adenosylmethionine, production of sugar phosphate, production of acetyl-CoA, production of propanoyl-CoA, production of oxyluciferin, production of guanosine-3′-diphosphate-5′-triphosphate, production of 5-phosphoribosyl-1-pyrophosphate, production of acyl-CoA, production of biotin-CoA, production of aminoacyl-tRNA, production of circular RNA, production of L-asparagine, production of L-asparatic acid, production of sugar nucleotide, and production of 3′-phosphoadenosine-5′-phosphosulfate. It should be easy for those skilled in the art to understand enzymatic reactions that generate a substance using ATP other than the foregoing reactions, by searching, for example, KEGG (http://www.genome.jp/kegg/).

The following description will discuss <1. Method of producing oxidized glutathione> and <2. Method of producing reduced glutathione> which are typical examples of production of a substance using ATP.

<1. Method of Producing Oxidized Glutathione>

One or more embodiments of the present invention provide a method of producing a substance, the method including the steps of:
(1) allowing L-glutamic acid and L-cystine to react with each other to produce oxidized γ-glutamylcysteine; and (2) allowing the oxidized γ-glutamylcysteine obtained from step (1) and glycine to react with each other to produce oxidized glutathione.

In one or more embodiments of the present invention, a method of producing oxidized glutathione is preferably a method disclosed in PCT International Publication No. WO 2016/002884.

In one or more embodiments of the present invention, the step (1) is represented by, for example, the following formula.

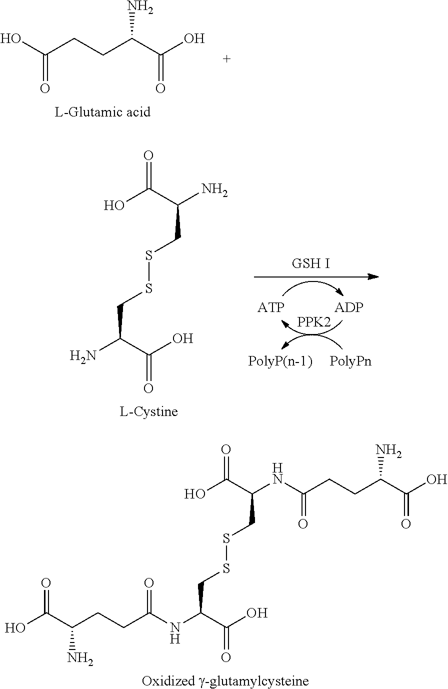 [SMILES: N[C@@H](CCC(=O)O)C(=O)O ; N[C@@H](CSSC[C@H](N)C(=O)O)C(=O)O ; "PPK2" ; N[C@@H](CCC(=O)N[C@@H](CSSC[C@H](NC(=O)CC[C@H](N)C(=O)O)C(=O)O)C(=O)O)C(=O)O]

The step (1) includes generating oxidized γ-glutamylcysteine by allowing L-cystine and L-glutamic acid to react with each other in the presence of GSHI and ATP.

The GSHI for use in the step (1) is not particularly limited, provided that the GSHI has the above-described activity. The origin of the GSHI is not particularly limited, and GSHI derived from a microorganism, an animal, a plant, or the like can be used. In one or more embodiments, GSHI derived from a microorganism is preferred. In one or more embodiments, for example, those derived from enteric bacteria such as Escherichia coli, those derived from bacteria such as coryneform bacteria, thermophilic bacteria/thermotolerant bacteria, psychrophilic bacteria/psychrotolerant bacteria, acidophilic bacteria/aciduric bacteria, basophilic bacteria/base-resistant bacteria, methylotroph, halogen-resistant bacteria, sulfur bacteria, and radiation-resistant bacteria, and those derived from eukaryotic microorganisms such as yeast are preferred.

In one or more embodiments of the present invention, the step (2) is represented by, for example, the following formula.

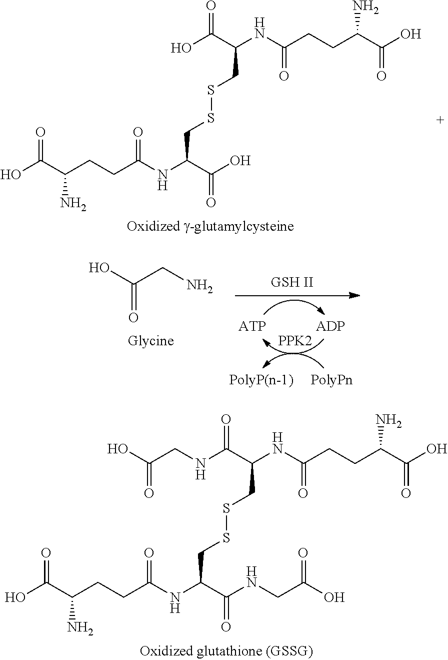 [SMILES: N[C@@H](CCC(=O)N[C@@H](CSSC[C@H](NC(=O)CC[C@H](N)C(=O)O)C(=O)O)C(=O)O)C(=O)O ; NCC(=O)O ; "PPK2" ; N[C@@H](CCC(=O)N[C@@H](CSSC[C@H](NC(=O)CC[C@H](N)C(=O)O)C(=O)NCC(=O)O)C(=O)NCC(=O)O)C(=O)O]

On the contrary, the step (2) includes generating oxidized glutathione by allowing the oxidized γ-glutamylcysteine and glycine to react with each other in the presence of GSHII and ATP.

The GSHII for use in the step (2) is not particularly limited, provided that the GSHII has the foregoing activity. The origin of the GSHII is not particularly limited, and GSHII derived from a microorganism, an animal, a plant, or the like can be used. In one or more embodiments, GSHII derived from a microorganism is preferred. In one or more embodiments, for example, those derived from enteric bacteria such as Escherichia coli, those derived from bacteria such as coryneform bacteria, thermophilic bacteria/thermotolerant bacteria, psychrophilic bacteria/psychrotolerant bacteria, acidophilic bacteria/aciduric bacteria, basophilic bacteria/base-resistant bacteria, methylotroph, halogen-resistant bacteria, sulfur bacteria, and radiation-resistant bacteria, and those derived from eukaryotic microorganisms such as yeast are preferred.

In one or more embodiments of the present invention, GSHF may be used instead of one of the GSHI and GSHII or instead of both of the GSHI and GSHII. GSHF is a bifunctional glutathione synthase that has both the functions of the two enzymes GSHI and GSHII, and is not particularly limited, provided that the GSHF can substitute the GSHI and GSHII. The origin of the GSHF is not particularly limited, and GSHF derived from a microorganism, an animal, a plant, or the like can be used. In one or more embodiments, GSHF derived from a microorganism is preferred. In one or more embodiments, for example, those derived from enteric bacteria such as Escherichia coli, those derived from bacteria such as coryneform bacteria, thermophilic bacteria/thermotolerant bacteria, psychrophilic bacteria/psychrotolerant bacteria, acidophilic bacteria/aciduric bacteria, basophilic bacteria/base-resistant bacteria, methylotroph, halogen-resistant bacteria, sulfur bacteria, radiation-resistant bacteria, and lactic acid bacteria, and those derived from eukaryotic microorganisms such as yeast are preferred. The GSHF is, for example, preferably GSHF derived from at least one selected from the group consisting of: Streptococcus bacteria such as Streptococcus agalactiae, Streptococcus mutans, Streptococcus suis, Streptococcus thermophilus, Streptococcus sanguinis, Streptococcus gordonii, and Streptococcus uberis; Lactobacillus bacteria such as Lactobacillus plantarum, Lactobacillus casei, Lactobacillus rhamnosus, Lactobacillus paracasei, Lactobacillus plantarum, and Lactobacillus fermentum; Desulfotalea bacteria such as Desulfotalea psychrophila; Clostridium bacteria such as Clostridium perfringens; Listeria bacteria such as Listeria innocua and Listeria monocytogenes; Enterococcus bacteria such as Enterococcus faecalis, Enterococcus faecium, and Enterococcus italicus; Pasteurella bacteria such as Pasteurella multocida; Mannheimia bacteria such as Mannheimia succiniciprodecens; Haemophilus bacteria such as Haemophilus somnus; Actinobacillus bacteria such as Actinobacillus succinogenes and Actinobacillus pleuropneumoniae; and Bacillus bacteria such as Bacillus cereus.

In one or more embodiments of the present invention, each of the foregoing ten types of PPK2 functions in a manner coupled with at least one selected from the group consisting of γ-glutamylcysteine synthase (GSHI), glutathione synthase (GSHII), and bifunctional glutathione synthase (GSHF).

A combination of any of the ten types of PPK2 and at least one selected from the group consisting of the GSHI, GSHII, and GSHF is not particularly limited and may be any combination, provided that oxidized glutathione can be produced with a high conversion rate. The GSHI, GSHII, and GSHF may be used in combination with different types of PPK2 or may each be used in combination with the same type of PPK2.

In one or more embodiments of the present invention, each of the enzymes PPK2, GSHI, GSHII, and GSHF may be (i) in the form of a live cell of an organism having a corresponding enzyme activity, (ii) in the form of a dead but undamaged cell of an organism having a corresponding enzyme activity, (iii) in the form in which the enzyme is present extracellularly, specifically, in the form of the foregoing cell of an organism which has been triturated, or (iv) in the form of a protein that has been isolated from the cell and purified. In one or more embodiments, it is preferable not to use live cells having the PPK2 activity. In one or more embodiments, it is more preferable to use neither live cells having the PPK2 activity nor undamaged dead cells.

In one or more embodiments of the present invention, the polyphosphoric acid mixture is preferably used (i.e., added) both in the steps (1) and (2) in view of the rate of conversion to oxidized glutathione; however, the polyphosphoric acid mixture may be used in only one of the steps (1) and (2).

<2. Method of Producing Reduced Glutathione>

One or more embodiments of the present invention provide a method of producing a substance, the method including the steps of:

(1) allowing L-glutamic acid and L-cysteine to react with each other to produce γ-glutamylcysteine; and

(2) allowing the γ-glutamylcysteine obtained from step (1) and glycine to react with each other to produce reduced glutathione.

In one or more embodiments of the present invention, a method of producing reduced glutathione is preferably a method disclosed in PCT International Publication No. WO2016/017631. WO2016/017631 discloses carrying out a reaction in a nitrogen atmosphere in order to prevent the oxidation of reduced glutathione; however, the production of reduced glutathione is not limited to a reaction in a nitrogen atmosphere.

In one or more embodiments of the present invention, the step (1) is represented by, for example, the following formula.

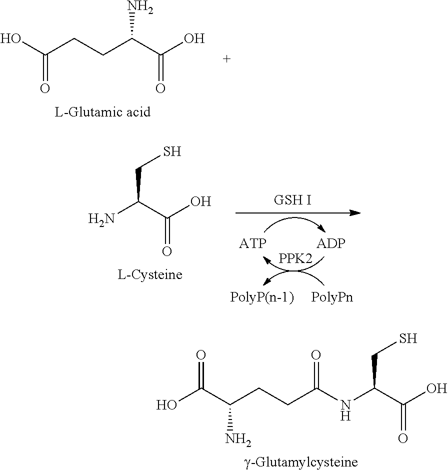 [SMILES: N[C@@H](CCC(=O)O)C(=O)O ; N[C@@H](CS)C(=O)O ; "PPK2" ; N[C@@H](CCC(=O)N[C@@H](CS)C(=O)O)C(=O)O]

The step (1) includes generating γ-glutamylcysteine by allowing L-cysteine and L-glutamic acid to react with each other in the presence of GSHI and ATP.

The GSHI for use in the step (1) is not particularly limited, provided that the GSHI has the above-described activity. In one or more embodiments, the origin of the GSHI is not particularly limited, and GSHI derived from a microorganism, an animal, a plant, or the like can be used. In one or more embodiments, GSHI derived from a microorganism is preferred. In one or more embodiments, for example, those derived from enteric bacteria such as Escherichia coli, those derived from bacteria such as coryneform bacteria, and those derived from eukaryotic microorganisms such as yeast are preferred.

In one or more embodiments of the present invention, the step (2) is represented by, for example, the following formula.

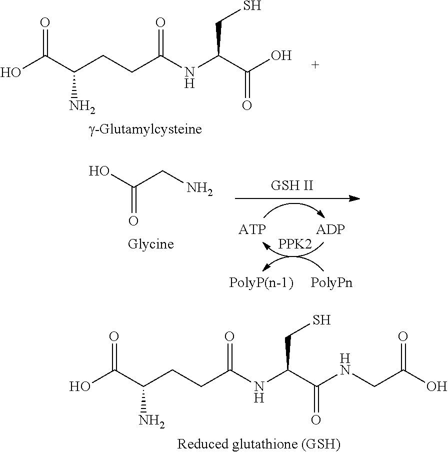 [SMILES: N[C@@H](CCC(=O)N[C@@H](CS)C(=O)O)C(=O)O ; NCC(=O)O ; "PPK2" ; N[C@@H](CCC(=O)N[C@@H](CS)C(=O)NCC(=O)O)C(=O)O]

On the contrary, the step (2) includes generating reduced glutathione by allowing the γ-glutamylcysteine and glycine to react with each other in the presence of GSHII and ATP.

The GSHII for use in the step (2) is not particularly limited, provided that the GSHII has the foregoing activity. The origin of the GSHII is not particularly limited, and GSHII derived from a microorganism, an animal, a plant, or the like can be used. In one or more embodiments, GSHII derived from a microorganism is preferred. In one or more embodiments, for example, those derived from enteric bacteria such as Escherichia coli, those derived from bacteria such as coryneform bacteria, and those derived from eukaryotic microorganisms such as yeast are preferred.

In one or more embodiments of the present invention, GSHF may be used instead of one of the GSHI and GSHII or instead of both of the GSHI and GSHII. The function, origin, and the like of the GSHF are the same as those described in the <1. Method of producing oxidized glutathione> section.

In one or more embodiments of the present invention, each of the foregoing ten types of PPK2 functions in a manner coupled with at least one selected from the group consisting of γ-glutamylcysteine synthase (GSHI), glutathione synthase (GSHII), and bifunctional glutathione synthase (GSHF).

A combination of any of the ten types of PPK2 and at least one selected from the group consisting of the GSHI, GSHII, and GSHF is not particularly limited and may be any combination, provided that reduced glutathione can be produced with a high conversion rate. The GSHI, GSHII, and GSHF may be used in combination with different types of PPK2 or may each be used in combination with the same type of PPK2.

In one or more embodiments of the present invention, each of the enzymes PPK2, GSHI, GSHII, and GSHF may be (i) in the form of a live cell of an organism having a corresponding enzyme activity, (ii) in the form of a dead but undamaged cell of an organism having a corresponding enzyme activity, (iii) in the form in which the enzyme is present extracellularly, specifically, in the form of the foregoing cell of an organism which has been triturated, or (iv) in the form of a protein that has been isolated from the cell and purified. In one or more embodiments, it is preferable not to use live cells having the PPK2 activity. In one or more embodiments, it is more preferable to use neither live cells having the PPK2 activity nor undamaged dead cells.

In one or more embodiments of the present invention, the polyphosphoric acid mixture is preferably used (i.e., added) both in the steps (1) and (2) in view of the rate of conversion into reduced glutathione; however, the polyphosphoric acid mixture may be used in only one of the steps (1) and (2).

Specifically, one or more embodiments of the present invention encompass the following subject matters.

[1] A method of producing a substance using ATP, wherein: ADP is generated from ATP during the method; the method is coupled with an ATP regeneration reaction in which a polyphosphate kinase 2 and polyphosphoric acid are allowed to react with the ADP to regenerate ATP; and the ATP used in the method includes the ATP regenerated by the ATP regeneration reaction, the method including using, as a substrate for the polyphosphate kinase 2, a polyphosphoric acid mixture that contains polyphosphoric acid molecules with a degree of polymerization of not less than 15 in an amount of not less than 48%.

[2] The method as set forth in [1], wherein the amount of the polyphosphoric acid molecules with a degree of polymerization of not less than 15, contained in the polyphosphoric acid mixture, is not less than 50%.

[3] The method as set forth in [1] or [2], wherein the polyphosphate kinase 2 is at least one selected from the group consisting of: polyphosphate kinase 2 derived from Pseudomonas aeruginosa; polyphosphate kinase 2 derived from Synechococcus sp. PCC6312; polyphosphate kinase 2 derived from Corynebacterium efficiens; polyphosphate kinase 2 derived from Kineococcus radiotolerans; polyphosphate kinase 2 derived from Pannonibacter indicus; polyphosphate kinase 2 derived from Deinococcus radiodurans K1; polyphosphate kinase 2 derived from Gulbenkiania indica; polyphosphate kinase 2 derived from Arthrobactor aurescens TC1; polyphosphate kinase 2 derived from Thiobacillus denitrificans ATCC25259; and polyphosphate kinase 2 derived from Pseudomonas fluorescens.

[4] The method as set forth in any of [1] to [3], wherein the polyphosphate kinase 2 functions in a manner coupled with at least one selected from the group consisting of γ-glutamylcysteine synthase, glutathione synthase, and bifunctional glutathione synthase.

[5] The method as set forth in any of [1] to [4], wherein the method is a method of producing oxidized glutathione or a method of producing reduced glutathione.

[6] The method as set forth in [5], wherein: the method is a method of producing oxidized glutathione; and the method includes the steps of: (1) allowing L-glutamic acid and L-cystine to react with each other to produce oxidized γ-glutamylcysteine; and (2) allowing the oxidized γ-glutamylcysteine obtained from step (1) and glycine to react with each other to produce oxidized glutathione.

In addition, it should be noted that configurations described in the above sections can also be applied in other sections as appropriate. The present invention is not limited to the foregoing embodiments, but can be altered by a skilled person in the art within the scope of the claims. One or more embodiments of the present invention also encompass, in its technical scope, any embodiment derived by combining technical means disclosed in differing embodiments. The following description will more specifically discuss one or more embodiments of the present invention with reference to Examples. However, the present invention is not limited to such Examples.

### EXAMPLES

### [Reference Example 1] Construction of Expression Vector for Polyphosphate Kinase

In accordance with information disclosed in PCT International Publication No. WO 2006/080313, the following sequence was chemically synthesized at Eurofins Genomics K.K.: a gene sequence which (i) codes for a polypeptide that is the same as polyphosphate kinase (NCBI Reference Sequence: WP_023109529) (amino acid sequence: SEQ ID NO:1, base sequence: SEQ ID NO: 11) derived from Pseudomonas aeruginosa except that 81 amino acids at the N terminus of the polyphosphate kinase are cut off and alanine at position 82 is substituted with methionine (initiation codon), (ii) which has been subjected to codon optimization so as to fit with an E. coli host and (iii) has an NdeI site added at the 5′ terminus of the gene sequence and an EcoRI site added at the 3′ terminus of the gene sequence. This gene was digested with NdeI and EcoRI, and inserted between NdeI and EcoRI restriction sites downstream of the lac promoter of a plasmid pUCN18 (a plasmid obtained by modifying T at position 185 of pUC18 [produced by Takara Bio Inc.] to A by PCR and thereby destroying the NdeI site and, in addition, modifying GC at positions 471 and 472 to TG and thereby introducing a new NdeI site). In this way, a recombinant vector pPPK was constructed.

### [Reference Example 2] Preparation of Recombinant Organism that Expresses Polyphosphate Kinase

E. coli HB101 competent cells (produced by Takara Bio Inc.) were transformed with the recombinant vector pPPK constructed in Reference Example 1, thereby obtaining a recombinant organism E. coli HB101 (pPPK). Furthermore, E. coli HB101 competent cells (produced by Takara Bio Inc.) were transformed with pUCN18, thereby obtaining a recombinant organism E. coli HB101 (pUCN18).

### [Reference Example 3] Expression of Polyphosphate Kinase Gene in Recombinant Organism

The two types of recombinant organism (E. coli HB101 [pUCN18] and E. coli HB101 [pPPK]) obtained in Reference Example 2 were each inoculated into 5 ml of 2×YT medium (1.6%/0 triptone, 1.0% yeast extract, 0.5% sodium chloride, pH7.0) containing 200 μg/ml of ampicillin, and cultured with shaking at 37° C. for 24 hours. Each of the culture solutions obtained through the culture was subjected to centrifugation and thereby bacterial bodies were collected, and the bacterial bodies were suspended in 1 ml of 50 mM Tris-HCl buffer (pH8.0). This was homogenized with use of a UH-50 ultrasonic homogenizer (produced by SMT), and then bacterial residues were removed by centrifugation. In this way, cell-free extracts were obtained.

Polyphosphate kinase activity was measured with use of these cell-free extracts. The polyphosphate kinase activity was measured in the following manner. 5 mM sodium metaphosphate (produced by Wako Pure Chemical Corporation), 10 mM ADP disodium salt (produced by Oriental Yeast Co., Ltd.), 70 mM magnesium sulfate (produced by Wako Pure Chemical Corporation), and the cell-free extract were added to 50 mM Tris-HCl buffer (pH8.0), allowed to react at 30° C. for 5 minutes, and generated ATP was quantified by HPLC. The enzymatic activity by which 1 μmol of ATP is generated per minute under these reaction conditions was defined as 1 U. The result was that the ATP-generating activity of E. coli HB101 (pUCN18) was not more than 5 U/mL.

### [Reference Example 4] Preparation of Polyphosphate Kinase

The E. coli HB101 (pPPK) obtained in Reference Example 2 was inoculated into 5 ml of 2×YT medium (1.6% triptone, 1.0% yeast extract, 0.5% NaCl, pH7.0) containing 200 μg/ml of ampicillin, and cultured with shaking at 37° C. for 24 hours. The enzymatic activity was measured by the method discussed in Reference Example 3, and found to be 120 U/mL. Next, bacterial bodies were collected by centrifugation, suspended in 2.5 ml of 50 mM Tris-HCl buffer (pH8.0), and homogenized ultrasonically to obtain an enzyme liquid (polyphosphate kinase liquid).

### [Production Example 1] Production of Oxidized Glutathione

Oxidized glutathione was produced through the following two steps: step (A) of producing oxidized γ-glutamylcysteine from L-glutamic acid and L-cystine; and step (B) of producing oxidized glutathione from the oxidized γ-glutamylcysteine and glycine (the oxidized γ-glutamylcysteine was produced by a partially modified version of the method disclosed in <Example 1> of PCT International Publication No. WO 2016/002884).

<Step (A)>

0.3629 g of sodium L-glutamate monohydrate (2.15 mmol), 0.3113 g of L-cystine dihydrochloride (0.99 mmol), 0.7079 g of magnesium sulfate heptahydrate, 0.0583 g of ATP (0.11 mmol), 0.8 g of sodium metaphosphate, and 12 g of distilled water were mixed together, and 0.8 g of 15 wt % aqueous sodium hydroxide solution was used to adjust the pH of the mixture to 7.5. To the resultant solution, 2 g of an E. coli K12-derived γ-glutamylcysteine synthase (GSHI) liquid was added, and a polyphosphate kinase liquid was added so that the total PPK2 activity in the reaction liquid would be 20 U/mL, and a reaction was started. The reaction was carried out at a temperature of 30° C. for 6 to 8 hours.

Note that the GSHI liquid was prepared in accordance with Test 1 and Test 4 of PCT International Publication No. WO 2016/002884.

The polyphosphate kinase liquid was prepared in the same manner as described in Reference Examples 1 to 4.

<Step (B)>

Next, 0.19 g of glycine (2.53 mmol), 2 g of glutathione synthase liquid, a predetermined amount of a polyphosphate kinase liquid, 0.21 g of magnesium sulfate heptahydrate, 0.04 g of ATP, and 0.92 g of aqueous sodium metaphosphate solution (36.2 wt %) were added to the above reaction liquid, and a reaction was started. Before the reaction, 1.1 g of 15 wt % aqueous sodium hydroxide solution was used to adjust the pH to 7.5. The reaction was carried out at a temperature of 30° C. for 8 hours. Then, the reaction was stopped and the reaction liquid was analyzed.

The GSHII used here is the modified glutathione synthase (V260A) disclosed in Laid-open publication of Japanese Patent Application, Tokugan, No. 2016-214073.

The polyphosphate kinase liquid was prepared in the same manner as described in Reference Examples 1 to 4.

### [Example 1] Production of Oxidized Glutathione Using Polyphosphoric Acid Mixture

A plurality of polyphosphoric acid mixtures, each of which would serve as a substrate for a polyphosphate kinase 2 in an ATP-regenerating system, were prepared, and production of oxidized glutathione was carried out. Each polyphosphoric acid mixture was prepared by: synthesizing polyphosphoric acid in accordance with a method usually used in this technical field; and obtaining a mixture containing the polyphosphoric acid. The production of oxidized glutathione was carried out in accordance with the method discussed in Production Example 1.

As a result, it was found that the rate of conversion to oxidized glutathione is high in a case where a specific polyphosphoric acid mixture is used.

In view of this, the polyphosphoric acid mixture, which achieved a high rate of conversion to oxidized glutathione, was analyzed for the degree of polymerization of polyphosphoric acid. The analysis was carried out under the following conditions.

<Conditions Under which Analysis was Carried Out>
Ion chromatograph
Model: ICS-2100 produced by Thermo Fisher Scientific
Columns: IonPac AG11, AS11 (4 mm×250 mm)
Eluent: KOH gradient
Eluent flow rate: 1.0 mL/min.
Sample injection volume: 25 pL
Column temperature: 35° C.
Detector: Conductometric detector

The amount for each degree of polymerization was determined by calculating the proportion of the area of a peak relative to the sum (100%) of the areas of all peaks.

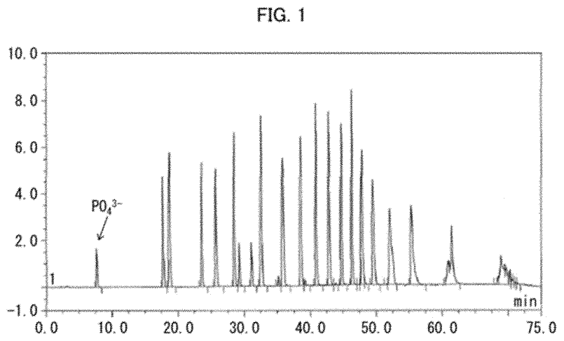

As a result, the distribution of polyphosphoric acid molecules with a high degree of polymerization in the polyphosphoric acid mixture was as follows (see FIG. 1).
Polyphosphoric acid molecules with a degree of polymerization of not less than 15: not less than 48%
Polyphosphoric acid molecules with a degree of polymerization of not less than 20: not less than 31%
Polyphosphoric acid molecules with a degree of polymerization of not less than 36: not less than 4%
Polyphosphoric acid molecules with a degree of polymerization of not less than 43: not less than 2%
Polyphosphoric acid molecules with a degree of polymerization of not less than 50: not less than 2%

The results showed that, in a case where such a polyphosphoric acid mixture containing a certain amount or more of polyphosphoric acid molecules with a high degree of polymerization is used as a substrate in a system in which ATP is regenerated by PPK2, it is possible to produce oxidized glutathione with a high conversion rate.

### [Example 2] Enzymatic Activity of Novel Polyphosphate Kinases

With regard to the following eight types of polyphosphate kinase (which are inferred from a database search to have polyphosphate kinase activity) and known DR PPK2, polyphosphate kinase liquids were prepared in the same manner as described in Reference Examples 1 to 4, and enzymatic activities were measured in the same manner as described in Reference Example 3.
PPK2 derived from Synechococcus sp. PCC6312 (Sy PPK2): SEQ ID NO:2
PPK2 derived from Corynebacterium efficiens (CE PPK2): SEQ ID NO:3
PPK2 derived from Kineococcus radiotolerans (KR PPK2): SEQ ID NO:4
PPK2 derived from Pannonibacter indicus (PI PPK2): SEQ ID NO:5
PPK2 derived from Deinococcus radiodurans K1 (DR PPK2): SEQ ID NO:6
PPK2 derived from Gulbenkiania indica (GI PPK2): SEQ ID NO:7
PPK2 derived from Arthrobactor aurescens TC1 (AA PPK2): SEQ ID NO:8
PPK2 derived from Thiobacillus denitrificans ATCC25259 (TD PPK2): SEQ ID NO:9
PPK2 derived from Pseudomonas fluorescens (PF PPK2) (PPK2 obtained by removing amino acids at positions 1 to 85 at the N terminus of native PF PPK2 (SEQ ID NO:10) and introducing a P86M mutation, and PPK2 obtained by removing amino acids at positions 1 to 86 at the N terminus of native PF PPK2 (SEQ ID NO:10) and introducing a G87M mutation)

As a result, all the eight types of novel polyphosphate kinase were found to have enzymatic activity. It was also confirmed that the known DR PPK2 has enzymatic activity.

The enzymatic activity of the PI PPK2 was 138 U/mL. This showed that the enzymatic activity of the PI PPK2 is higher than that of the PNDK (120 U/mL (see Reference Example 4)).

Note that, in a case where the same test as described above was carried out with use of a polyphosphoric acid mixture solution that had been left to stand at room temperature for 13 days from its preparation, the enzymatic activity of the PI PPK2 was 730% of that in a case where a polyphosphoric acid mixture solution immediately after the preparation was used (assuming that the enzymatic activity of this case is 100%).

### [Example 3] Production of Oxidized Glutathione Using Various Types of Polyphosphoric Acid Mixture

Production of oxidized glutathione was carried out in accordance with the method discussed in Production Example 1.

The following three types of polyphosphate kinase were used (used enzyme was the same between the step (A) and the step (B)).
PPK2 derived from Pseudomonas aeruginosa (PNDK) (a protein obtained by cutting off 81 amino acids at the N terminus of wild-type PNDK (SEQ ID NO:1) and substituting alanine at position 82 with methionine (initiation codon))
PPK2 derived from Pannonibacter indicus (PI PPK2)
PPK2 derived from Synechococcus sp. PCC6312 (Sy PPK2)

With regard to the PNDK, PI PPK2, and Sy PPK2, tests were carried out under the conditions shown in Table 1 below. The enzymatic activity in a reaction liquid was adjusted by adding a certain amount of culture solution (bacterial bodies) based on the enzymatic activity per culture solution.

TABLE 1

| | | Duration of storage of | |
|---|---|---|---|
| Name of test | Name of enzyme | metaphosphoric acid | |
| Test A | PNDK | 0 days (used immediately | |
| | | after preparation) | |
| Test B | | 5 | days |
| Test C | PI PPK2 | 1 | day |
| Test D | | 1 | day |
| Test E | | 1 | day |
| Test F | | 1 | day |
| Test G | | 5 | days |
| Test H | | 5 | days |
| Test I | Sy PPK2 | 0 days (used immediately | |
| | | after preparation) | |
| Test J | | 8 | days |

The amount of each polyphosphate kinase liquid added in the step (B) was an amount that achieves a corresponding PPK2 activity in the reaction liquid as shown in Table 2.

TABLE 2

| | Enzymatic activity |
|---|---|
| Name of test | in reaction liquid (U/mL) |
| Test A | 61 |
| Test B | 86 |
| Test C | 60 |
| Test D | 120 |
| Test E | 47 |
| Test F | 38 |
| Test G | 45 |
| Test H | 22 |
| Test I | 31 |
| Test J | 63 |

On the basis of above, production of oxidized glutathione was carried out. The results are shown in Table 3.

TABLE 3

| | Rate of conversion to |
|---|---|
| Name of test | oxidized glutathione (%) |
| Test A | 99 |
| Test B | 63 |
| Test C | 99.5 |
| Test D | 99.6 |
| Test E | 99.8 |
| Test F | 99.9 |
| Test G | 57 |
| Test H | 44 |
| Test I | 71 |
| Test J | 22 |

The above results showed that, in cases of all types of polyphosphate kinase, use of an aqueous metaphosphoric acid solution after long-term storage results in a reduction in rate of conversion to oxidized glutathione (this is apparent from a comparison between test A and test B on PNDK, a comparison between tests C-F and tests G-H on PI PPK2, and a comparison between test I and test J on Sy PPK2).

Specifically, the results were as follows: the rate of conversion to oxidized glutathione was lower in cases where an aqueous metaphosphoric acid solution after long-term storage was used than in cases where an aqueous metaphosphoric acid solution after short-term storage was used, although the PPK2 enzymatic activity in the reaction liquid was high in the former case (this was apparent from a comparison between test A and test B on PNDK, a comparison between test F and test G on PI PPK2, and a comparison between test I and test J on Sy PPK2). A reason therefor is inferred to be that, although the PPK2 activity in the reaction system was high enough in tests B, G, and J, the metaphosphoric acid serving as a substrate for polyphosphate kinase was degraded during the storage and became insufficient.

### [Example 4] Changes in Composition (Degree of Polymerization) of Polyphosphoric Acid Mixture: (1)

In view of the results of Example 3, the following test was carried out to confirm that the composition of a polyphosphoric acid mixture changes over time during storage.

Specifically, water was added to sodium metaphosphate to prepare 50 w/v % sodium metaphosphate. This was used as sample 1. After 13 days from the preparation of the sample 1, another 50 w/v % sodium metaphosphate was prepared (sample 2). On the day on which the sample 2 was prepared, the samples 1 and 2 were analyzed for the degree of polymerization of metaphosphoric acid contained therein. The analysis was carried out under the following conditions.

<Conditions Under which Analysis was Carried Out>
Ion chromatograph
Model: ICS-2100 produced by Thermo Fisher Scientific
Columns: IonPac AG11, AS11 (4 mm×250 mm)
Eluent: KOH gradient
Eluent flow rate: 1.0 mL/min.
Sample injection volume: 25 pL
Column temperature: 35° C.
Detector: Conductometric detector

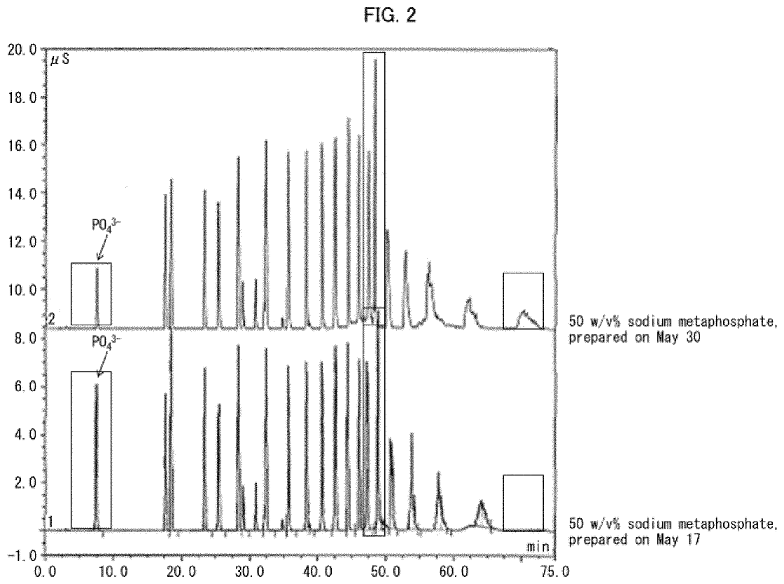

The results are shown in FIG. 2. As is clear from FIG. 2, the sample 1, which had been left to stand for 13 days after the preparation, had a degreased amount of metaphosphoric acid molecules with a high degree of polymerization, as compared to the sample 2 which was analyzed immediately after the preparation. This result shows that, if an aqueous metaphosphoric acid solution is left to stand at room temperature, metaphosphoric acid molecules with a higher degree of polymerization are degraded first.

The combination of the results of this example and the results of Example 3 suggests that, in order to carry out a conversion from ADP to ATP efficiently by polyphosphate kinase, it is not only necessary that the polyphosphate kinase have sufficient level of enzymatic activity but also necessary that metaphosphoric acid serving as a substrate for the polyphosphate kinase be in an appropriate condition (specifically, metaphosphoric acid molecules with a high degree of polymerization be present).

### [Example 5] Changes in Composition (Degree of Polymerization) of Polyphosphoric Acid Mixture: (2)

The following test was carried out to confirm that the composition (degree of polymerization) of polyphosphoric acid mixture changes during production of oxidized glutathione.

Specifically, the same test as test G of Example 3 was carried out, a reaction liquid immediately after the completion of the step (a) and a reaction liquid immediately after the completion of the step (b) were recovered, and the composition (degree of polymerization) of polyphosphoric acid contained in each reaction liquid was checked. The composition (degree of polymerization) was analyzed in accordance with the method discussed in Example 4.

The sample 2 of Example 4 (polyphosphoric acid mixture immediately after preparation) was used as a polyphosphoric acid mixture before consumed (i.e., before used) by PPK2.

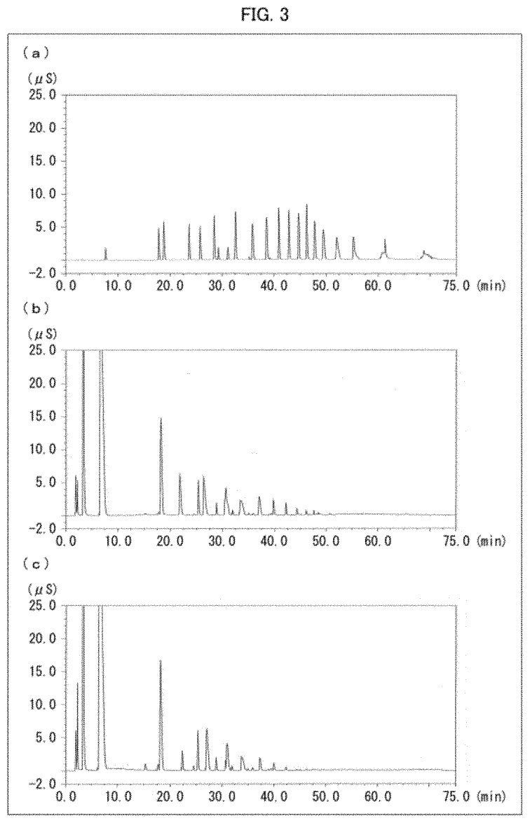

The results are shown in FIG. 3. Panel (a) of FIG. 3 shows the composition (degree of polymerization) of a polyphosphoric acid mixture immediately after preparation, panel (b) of FIG. 3 shows the composition (degree of polymerization) of a polyphosphoric acid mixture after completion of the step (A), and panel (c) of FIG. 3 shows the composition (degree of polymerization) of a polyphosphoric acid mixture after completion of the step (B).

The results show that, in the reaction in which ATP is regenerated by PPK2, polyphosphoric acid molecules with a high degree of polymerization are used first.

One or more embodiments of the present invention make it possible to produce a substance using ATP with a high conversion rate at low cost, and is therefore usable in the fields of, for example, production of oxidized glutathione and production of reduced glutathione.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A method of producing a substance, comprising synthesizing a molecule at least by mixing substrates, a synthase, adenosine triphosphate (ATP), a polyphosphate kinase 2, and a polyphosphoric acid mixture,
wherein the polyphosphoric acid mixture comprises 50% or more of polyphosphoric acid with a degree of polymerization of not less than 15,
wherein adenosine diphosphate (ADP) is generated from the ATP during the synthesis,
wherein the synthesis is coupled with an ATP regeneration reaction in which the ATP is regenerated by the polyphosphate kinase 2 from the ADP and the polyphosphoric acid, and
wherein the polyphosphate kinase 2 is polyphosphate kinase 2 having a sequence identity of not less than 80% to at least one selected from the group consisting of: polyphosphate kinase 2 derived from Pseudomonas aeruginosa; polyphosphate kinase 2 derived from Synechococcus sp. PCC6312; polyphosphate kinase 2 derived from Corynebacterium efficiens; polyphosphate kinase 2 derived from Kineococcus radiotolerans; polyphosphate kinase 2 derived from Pannonibacter indicus; polyphosphate kinase 2 derived from Deinococcus radiodurans K1; polyphosphate kinase 2 derived from Gulbenkiania indica; polyphosphate kinase 2 derived from Arthrobactor aurescens TC1; polyphosphate kinase 2 derived from Thiobacillus denitrificans ATCC25259; and polyphosphate kinase 2 derived from Pseudomonas fluorescens.

2. The method according to claim 1,
wherein the polyphosphate kinase 2 functions in a manner coupled with the synthase, and
wherein the synthase is at least one selected from the group consisting of γ-glutamylcysteine synthase, glutathione synthase, and bifunctional glutathione synthase.

3. The method according to claim 1, wherein the method comprises producing oxidized glutathione or reduced glutathione.

4. The method according to claim 1, further comprising synthesizing oxidized glutathione by reacting the molecule with glycine,
wherein the molecule is oxidized γ-glutamylcysteine, and
wherein the substrates comprise L-glutamic acid and L-cystine reacting with each other to produce the oxidized γ-glutamylcysteine.

5. The method according to claim 1, wherein the polyphosphate kinase 2 is polyphosphate kinase 2 derived from Pannonibacter indicus.


SEQUENCE LISTING (20 sequences)

SEQ ID NO: 1 - protein, 357 aa, Pseudomonas aeruginosa
MSEEPTVSPPSPEQPAAQPAKPARPAARRAPRKPATRRPRVASPAQKAREEIQAISQKPVALQVASAPHGSSEDSTSASLPANYPYHTRMRRNEYEKAKHDLQIELLKVQSWVKETGQRVVVLFEGRDAAGKGGTIKRFMEHLNPRGARIVALEKPSSQEQGQWYFQRYIQHLPTAGEMVFFDRSWYNRAGVERVMGFCSPLQYLEFMRQAPELERMLTNSGILLFKYWFSVSREEQLRRFISRRDDPLKHWKLSPIDIKSLDKWDDYTAAKQAMFFHTDTADAPWTVIKSDDKKRARLNCIRHFLHSLDYPDKDRRIAHEPDPLLVGPASRVIEEDEKVYAEAAAAPGHANLDIPA

SEQ ID NO: 2 - protein, 296 aa, Synechococcus sp.
MAELDITAAPLEAQTEGPGKKKKAKDKKKALPETPKPSKLDRDFYDKELARLQVELVKMQYWVKHAGLKIVIIFEGRDAAGKGGMIKRISAPLNPRGCRIVALGTPSDREKTQWYFQRYVEHLPGAGEIVMFDRSWYNRAGVEWVMGFCTEAQYNEFMDSCPQFERMLVKSGIILIKYWFSVSDDEQERRFQARILEPAKRWKISPMDIESRDRWVDYSKAKDAMLAHTNIPEAPWFTVEADDKRRAHLNCISHLLSKIPYEDITPPAIDLPPRRPAPEDYVRPPINEQFFVPSIY

SEQ ID NO: 3 - protein, 351 aa, Corynebacterium efficiens
MNKMENAPMPTFGKELPKLDNKAYKKELKRLQAELVEMQQWVVETGTRVVIVMEGRDAAGKGSAIKRITQYLNPRTARIEALPTPTSREKGQWYFQRYVEKLPAAGEIVIFDRSWYNRAGVERVMGFCTSQEYRRFLHQAPIFERLLVEDGIHLRKYWFSVSDEEQLARFHSRLSDPLRRWKLSTIDLHSITRWEDYSRAKDEMFIHTDIPSAPWYTVESEEKKRSRINVISHILSTIPYEKIDRPLPEIPERPVREGEYIRPPRNEFRYVPDVAACLEEHRVAAAREKAKAEAKAREEAERALAAEKVKAAKKAKKIRKAQKAKAAKKAKKAAGKAKAVKKTGKSGKGGK

SEQ ID NO: 4 - protein, 296 aa, Kineococcus radiotolerans
MPHVQLTPDLGMTVRDDEDEPELLTPDGNVVDTWREDYPYDERLDRKEYDAEKRLLQIELLKLQRWLKASGERIVVLCEGRDAAGKGGTIKRFMEHLNPRGARVVALEKPSERESTQWYFQRYVQHLPAAGEFVLFDRSWYNRAGVERVMGFASPAEYDRFVAQAPLFEKMLVDDGIHLVKFWFSVTRAEQRTRFLIRQIDPVRQWKLSPMDLESLDRWDEYTAAKEAMFATTDTDVAPWTVVKTNDKKRARLAAMRHVLARFDYDGKDPEVVGVPDPLLVVHARTILEADRRPAS

SEQ ID NO: 5 - protein, 367 aa, Pannonibacter indicus
MDDRTVTRKPRGTRPAPAADAVLPVTADSAEAEATPSGVAETPAEAVTAPQPGTEPVAAPEAALEPAIAPVAAAPRKTLAEMRHDPAAIHELFESGKYPYATPMRRAPYEQRKAKLQAELLKAQRWIKETGQRVVILFEGRDAAGKGGTIKRFMEHLNPRGATVVALQKPTEQEASQWYFQRYINHLPSGGEMVLFDRSWYNRAGVERVMGFCSASQYLEFMRQCPEIERMMVRDGIRLFKYWFSVSREEQRRRFMERQTDPLKQWKLSPIDIASLDKWDDYTEAKEAMFFYTDTADAPWTIVKSDDKKRARLNCMEHFLHSLPYPDKDIHLVGVPDPLIVGAAHHVISHDTHILGKALHPEMKPAG

SEQ ID NO: 6 - protein, 266 aa, Deinococcus radiodurans
MDIDNYRVKPGKRVKLSDWATNDDAGLSKEEGQAQTAKLAGELAEWQERLYAEGKQSLLLILQARDAAGKDGAVKKVIGAFNPAGVQITSFKQPSAEELSHDFLWRIHQKAPAKGYVGVFNRSQYEDVLVTRVYDMIDDKTAKRRLEHIRHFEELLTDNATRIVKVYLHISPEEQKERLQARLDNPGKHWKFNPGDLKDRSNWDKFNDVYEDALTTSTDDAPWYVVPADRKWYRDLVLSHILLGALKDMNPQFPAIDYDPSKVVIH

SEQ ID NO: 7 - protein, 350 aa, Gulbenkiania indica
MNEKPLIPAPTDETAASSEQAAPDTPAAAAASARSARSRRRRPPTETSKAHDESVQAIEAAQPGPVALEVALAPGGSTEDSTTAPLPACYPYRTRMRRPEYERLKAELQIELLKVQNWIKETGQRVIVLFEGRDAAGKGGTIKRFMEHLNPRGARVVALEKPTEVERGQWYFQRYIQHFPTAGEIVFFDRSWYNRAGVERVMGFCTPNEYLEFMRQAPELERMLVNSGIRLFKFWFSVSREEQLRRFIARRDDPLKHWKLSPIDIQSLDKWDEYTAAKQSMFFHTDTADAPWTVIKSDDKKRARINCIRHFLHQLPYPDKNPRVACQPDPLLVGNASKVLEPHETQVLTF

SEQ ID NO: 8 - protein, 314 aa, Arthrobacter aurescens
MTEASPSSPTGTSLDDWWVRDNLRETIDHLVELGYTISGGQGEDPDLIDPGGSAVETWNEDYPYQQRMTRDEYEIEKYRLQIELLKFQYWGQDLGLKHVIVFEGRDAAGKGGTIKRFTEHLDPRSARTVALAKPSDREQGQWYFQRYIQQFPTAGEIVMFDRSWYNRANVERVMGFCTDDEYDTFMGQAPVFEKMLVDAGIHVTKFWFSVTRQEQRTRFAIRQIDPVRRWKLSPMDLASLDRWDEYTDAKERTFLHTDSDHAPWITIKSNDKKRARINAMRYFLNQFDYEDKDTSVVYDADPLILRRGRDAVGD

SEQ ID NO: 9 - protein, 269 aa, Thiobacillus denitrificans
MKPRDTRVKPGSRVSLADWHTDGDAFVGKDKAASMARLDDDRVRLEELQELLYAEGKHRLLVVLQAMDTAGKDSTIRHVFRGVDPLGVRVANFGVPSTHELRHDYLWRVHPHVPASGEIAIFNRSHYEDVLVPRVNGAIGHAECERRYRQINDFERMLSETGTTIRKFYLHISKDEQKKRLEARRDTPKKRWKFQPGDLAVRAQWDDYRAAYDALLSATSTRHAPWHVVPADDKLARNLIVSALLIEALEGLDMRYPEPVAGVAGTPII

SEQ ID NO: 10 - protein, 362 aa, Pseudomonas fluorescens
MSEESTALPLPPAPVQKAPASTSPASTKAAPRKAATPRPRRPRTTKAAPVKAAEAEISAISQKPMALQVANAPRGSNEDSVSAALPGNYPYRNRMRRAEYEKAKNELQIELLKVQSWVKETGQRIVVLFEGRDAAGKGGTIKRFMEHLNPRGARIVALEKPSEQEKGQWYFQRYIQHLPTAGEMVFFDRSWYNRAGVERVMEFCSPLQYLEFMRQTPELERMLCNSGILMFKFWFSVNREEQLRRFISRRDDPLKHWKLSPIDIKSLDKWDEYTAAKQAMFFHTDTADAPWTVIKSDDKKRARINCIRHFLHELDYPGKDLKVAHAPDPLLVGRASRGLEEDERTQAQAATDAGATKLALSA

SEQ ID NO: 11 - DNA, 1074 bp, Pseudomonas aeruginosa
atgagcgaagaacccactgtcagtcccccctcccccgagcaacccgccgcgcagccggccaagccggcccggccagccgcccgccgcgccccgcgcaagccggcgacccgccgcccgcgagtggccagcccggcgcagaaggcccgcgaggagatccaggcaatcagccagaagccggtggccctgcaggtcgccagtgcgccccacggcagcagcgaggacagcacctcggcgagcctgccggcgaactatccctatcacacgcggatgcgccgcaacgagtacgagaaggccaagcacgacctgcagatcgaactgctcaaggtgcagagctgggtgaaggagaccggccagcgcgtggtggtcctgttcgaaggccgcgacgccgccggcaagggcggcaccatcaagcgcttcatggaacacctgaacccgcgcggcgcgcggatcgtagccttggagaaaccgtcctcccaggagcagggccagtggtatttccagcgctacatccaacatctgcccaccgccggcgagatggtcttcttcgaccgctcctggtacaaccgcgccggcgtcgagcgggtcatgggcttctgttcgccgctgcaatacctggagttcatgcgccaggcgccggagctggagcgcatgctcaccaacagcggcatcctgctgttcaagtactggttctcggtgagccgcgaggaacaactgcggcgcttcatctcgcgccgcgacgatccgctcaagcactggaagctgtcgcccatcgacatcaagtctctggacaagtgggacgactacaccgccgccaagcaggcgatgttcttccataccgacaccgccgacgcgccgtggacggtcatcaagtccgacgacaagaagcgcgcgcgactcaactgcatccgccacttcctgcactcgctggactacccggacaaggaccggcgcatcgcccatgagcccgacccgttgctggtggggccggcctcgcgggtgatcgaggaggacgagaaggtctacgccgaggcggccgccgcgccgggccacgcgaacctggatatcccggcctga

SEQ ID NO: 12 - DNA, 903 bp, Synechococcus sp.
catatggccgagctggacattactgcggcaccgttagaggcccaaaccgaaggtcccggcaagaagaaaaaagcgaaagataagaagaaagcgttgccggaaaccccgaaaccgtccaaactggatcgggatttctacgacaaagaactggcacgccttcaggtagaactggtgaaaatgcagtactgggtgaaacacgctggcttaaaaatcgtgattatctttgaaggtcgtgatgcagctggcaaagggggaatgatcaaacgcatttcagcgccgttgaatccgcgtggatgtcgcattgtggctctgggtactccaagtgatcgcgagaaaacgcagtggtactttcagcgctatgtcgagcatctacctggtgcaggcgaaatagtcatgttcgatcgtagctggtacaatagggcaggtgtggaatgggtgatgggcttttgcaccgaagcgcagtataacgagttcatggatagttgtccacagtttgagcgtatgctggtcaaatccgggataatcctgattaagtactggtttagcgtttcggatgacgaacaagaacgccgctttcaagctcgcattctggaaccggccaaacgctggaagatctctccgatggacatcgaatcacgggatcgttgggttgactattcgaaagccaaagatgccatgcttgcgcataccaatattccggaagccccatggtttacggttgaagcggacgataaacgacgtgcgcatctgaactgcatctctcacttactcagcaaaatcccgtatgaggacattacacctcccgccattgatctcccaccgcgtagacctgcgcctgaagattatgtacgtccgccgattaacgaacagttcttcgttcccagcatctattaataagaattc

SEQ ID NO: 13 - DNA, 1068 bp, Corynebacterium efficiens
catatgaataaaatggaaaacgctccgatgccgacgtttggcaaagaactgcccaaactggacaacaaagcgtacaaaaaggagctcaaacgtctgcaagcagagctggtggaaatgcagcaatgggttgtggaaacgggtactcgcgtcgtgattgtgatggaaggtcgagatgcggccggaaaaggctccgcgattaagcgcatcacccagtacctgaatccgcgtacagcccgtattgaggcattaccgactcctacctctcgcgaaaaaggtcagtggtatttccagaggtatgtagagaagttacctgctgcgggtgagattgtgatctttgatcgcagctggtacaatcgcgccggcgttgaacgtgttatgggcttttgcacgagtcaggaatatcgccgctttttgcatcaggcaccgatcttcgaacgcttgttagtcgaagatggcatacatctccgcaagtattggttctctgtatcggatgaagaacaacttgcccgctttcatagccggctgagtgatccgctgcgtcgttggaaactgtcgacgatagatctgcacagcattacccgttgggaggactactcacgtgcgaaagacgagatgttcattcacaccgatattcccagcgcaccatggtatacagtggaatcggaggagaaaaaacgcagtcgcatcaacgtaattagccacatcctttcaaccattccgtacgagaagattgaccgtccgttgccggaaatcccagaacgcccagtgcgggaaggggagtatatccgtccacctcggaacgaatttcgctatgtcccggatgttgccgcttgtctggaagaacatcgagttgcagcagcgcgtgaaaaagcaaaagcagaagctaaagcaagagaagaagcggaacgtgcgctagccgcggaaaaagtgaaagcggccaaaaaggcgaaaaagatccgtaaagcccagaaggccaaagctgcgaaaaaagccaaaaaagctgccggtaaggcgaaagcggtcaaaaaaaccggcaaatccgggaaaggcggaaaataataagaattc

SEQ ID NO: 14 - DNA, 903 bp, Kineococcus radiotolerans
catatgccgcatgttcagttgactccggatctgggtatgacagttcgcgatgatgaggatgaacctgaactgcttacgccagatggcaacgtggtagatacctggcgcgaagattacccgtacgacgaacgtctggatcgtaaggagtatgatgccgagaaacgtctgctgcaaatcgagttgctcaaacttcagcgttggttgaaagcctctggtgaacgcatcgtagtgctttgcgaaggacgcgacgctgcgggcaaaggcggcacgatcaaacgtttcatggaacacctgaatccgcgtggtgcccgggtagttgcactggagaaaccgtcggaacgtgaaagcacccagtggtactttcagcgctatgtccagcacttacccgcagctggcgagtttgtgctgttcgatcggagctggtataatcgcgctggtgtagaacgtgtcatgggttttgcgagtcccgcagaatacgaccgcttcgttgcccaagccccgttattcgagaaaatgctcgttgatgatgggattcacctggtgaagttctggttttccgtgactcgcgcggaacaacgtacccgctttctgattcgccagattgacccggtgcgccagtggaaactgtcaccaatggacctggaaagcctggaccgctgggatgaatataccgcggcgaaagaagcgatgtttgccaccaccgatacggatgtggcaccgtggacagtcgtcaagaccaacgacaagaaacgcgcacgtttagcggcaatgcgccatgtcttagcgcgctttgactatgatgggaaagacccagaagtggtgggagttccggaccctctgctggtggttcatgctcggacgattctcgaagcggatcgtcgccctgcctcgtaatgagaattc

SEQ ID NO: 15 - DNA, 1116 bp, Pannonibacter indicus
catatggacgaccgtaccgtgacccgcaaaccacgcggaacacggccagcaccagctgcggatgccgtgttgccggtgaccgccgatagcgccgaagctgaagcgacccctagcggcgttgctgaaacgccggcggaggcagtgactgcaccgcaacctgggacagaaccggtcgccgctcctgaagccgcgttagaaccggcgattgcccctgttgcagcagcaccccgcaaaaccctggctgaaatgcgccatgatccggcggcgattcacgaactgtttgagtctggcaagtatccgtatgcgaccccgatgcgtcgcgctccgtatgaacagcgcaaagcgaaactccaagccgaactgctgaaagcccaacggtggatcaaagaaacgggtcaacgcgtggtcatcctgttcgagggccgtgatgcggcgggcaaaggtgggaccatcaaacgctttatggagcatctgaatccccgtggtgcaactgtggttgcgttacagaaacccacggaacaggaagccagccagtggtactttcagcgttacattaaccatctcccgtcaggcggtgaaatggtcctgtttgaccgttcgtggtacaatcgggcaggagttgagcgcgtaatgggcttttgcagtgcgtcgcagtatctcgagtttatgcgtcaatgtccggaaattgagcgcatgatggtccgtgatgggattcgcctgtttaagtactggttcagcgtgagtcgcgaagaacagcgtcgccgtttcatggagcgtcagaccgatccgttgaagcagtggaagttatcaccaatcgatattgcctccctggataaatgggatgattacaccgaagcgaaagaagccatgttcttctatacggacacagccgacgcgccgtggactatcgtgaaatccgacgataagaaacgcgcgcgcctgaactgcatggagcacttccttcattctcttccgtatcccgacaaagacattcacctggttggtgtaccagatcctttgatcgtaggtgcagctcatcacgtcatttcgcacgatacgcatattctgggcaaagccctgcatccggaaatgaaaccggcaggctaatgagaattc

SEQ ID NO: 16 - DNA, 813 bp, Deinococcus radiodurans
catatggatatcgacaactatcgcgtgaaacctggtaaacgggtgaaactgtcggattgggcaacgaacgatgatgcaggcttgtccaaagaagaaggtcaggcacaaacggccaaacttgcgggtgaattggctgaatggcaggaacgcctttatgcggaaggcaagcaaagtttactgctgattctgcaagcacgcgatgcggctgggaaagatggtgccgttaagaaggtgattggcgcgtttaaccctgccggagttcagatcaccagcttcaaacagccgagtgcggaagaactgtcacatgacttcttatggcgcattcatcagaaagctcccgcgaaagggtacgtaggcgtatttaaccgttctcagtatgaagatgtcttggttactcgcgtctatgacatgatcgatgacaagaccgctaaacgtcgcctggagcatattcggcactttgaggaactgctgacggataatgccacacgtatcgtgaaagtctacctccatattagcccggaagagcagaaagagcgtctgcaagcccgtcttgacaatccgggaaaacactggaaatttaacccaggcgacctgaaagaccgctccaattgggacaaattcaacgatgtctacgaggatgcgctgactacctctaccgatgatgcgccgtggtatgtagtgccagcagatcgcaaatggtatcgtgacttagtgctctcgcatattctgctgggtgccctcaaggatatgaatccgcagtttcccgcgatcgattacgacccgagcaaagttgtgattcactgataagaattc

SEQ ID NO: 17 - DNA, 1056 bp, Gulbenkiania indica
atgaacgaaaaaccgctgattccggccccgaccgatgaaaccgcggccagcagcgaacaggccgcaccggataccccggcggccgcggccgcgagcgcccgtagcgcgcgcagccgtcgccgtcgcccgccgaccgaaaccagcaaagcccatgatgaaagcgtgcaggcgattgaagcggcccagccgggtccggtggccctggaagttgcactggccccgggcggtagcaccgaagatagcaccaccgccccgctgccggcgtgctatccgtatcgtacccgtatgcgtcgtccggaatatgaacgcctgaaagcggaactgcaaattgaactgctgaaagttcagaactggattaaagaaaccggccagcgcgtgatcgttctgtttgaaggtcgcgatgccgcgggtaaaggcggcaccatcaaacgttttatggaacatctgaatccgcgtggcgcccgtgtggttgcgctggaaaaaccgaccgaagtggaacgcggccagtggtattttcagcgttatattcagcattttccgaccgccggcgaaatcgtgtttttcgatcgtagctggtataaccgcgcgggcgtggaacgtgttatgggtttttgtaccccgaatgaatatctggaatttatgcgccaggccccggaactggaacgtatgctggtgaacagcggtattcgcctgtttaaattttggtttagcgttagccgtgaagaacagctgcgtcgctttatcgcgcgtcgcgatgatccgctgaaacattggaaactgagcccgattgatatccagagcctggataaatgggatgaatataccgccgcgaaacagagcatgtttttccataccgataccgccgatgcgccgtggaccgtgattaaaagcgatgataaaaaacgtgcgcgcattaattgcatccgccattttctgcatcagctgccgtatccggataaaaacccgcgtgttgcctgtcagccggacccgctgctggttggcaatgcgagcaaagttctggaaccgcatgaaacccaggttctgaccttttaataa

SEQ ID NO: 18 - DNA, 948 bp, Arthrobacter aurescens
atgaccgaagcgagcccgagcagcccgaccggcaccagcctggatgattggtgggttcgtgataacctgcgcgaaaccattgatcatctggtggaactgggctataccatcagcggcggtcagggtgaagatccggatctgattgatccgggcggtagcgccgttgaaacctggaatgaagattatccgtatcagcagcgtatgacccgcgatgaatacgaaatcgaaaaataccgcctgcaaatcgaactgctgaaatttcagtattggggccaggatctgggtctgaaacatgtgattgtttttgaaggtcgtgatgcggccggtaaaggcggcaccatcaaacgttttaccgaacatctggacccgcgtagcgcccgtaccgttgccctggcaaaaccgagcgatcgcgaacagggccagtggtattttcagcgttatattcagcagtttccgaccgcgggtgaaatcgtgatgtttgatcgtagctggtataaccgcgccaatgtggaacgtgttatgggcttttgcaccgatgatgaatatgatacctttatgggtcaggcgccggtgtttgaaaaaatgctggttgatgccggtatccatgtgaccaaattttggtttagcgttacccgccaggaacagcgtacccgctttgcgattcgtcagatcgatccggtgcgtcgctggaaactgagcccgatggatctggcgagcctggatcgctgggatgaatataccgatgccaaagaacgtacctttctgcataccgatagcgatcatgccccgtggattaccatcaaaagcaacgataaaaaacgtgcgcgcattaacgccatgcgctattttctgaaccagttcgattacgaagataaagataccagcgtggtttatgatgccgatccgctgattctgcgtcgcggtcgtgatgcagtgggtgattaataa

SEQ ID NO: 19 - DNA, 813 bp, Thiobacillus denitrificans
atgaaaccgcgtgatacccgcgtgaaaccgggcagccgtgttagcctggcggattggcataccgatggcgatgcctttgtgggtaaagataaagcggccagcatggcgcgtctggatgatgatcgtgttcgcctggaagaactgcaagaactgctgtatgccgaaggcaaacatcgcctgctggttgtgctgcaagcgatggataccgccggtaaagatagcaccattcgtcatgtgtttcgcggcgttgatccgctgggtgtgcgcgttgcgaactttggcgtgccgagcacccatgaactgcgtcatgattatctgtggcgtgtgcatccgcatgttccggccagcggtgaaattgccatctttaaccgtagccattatgaagatgtgctggttccgcgcgttaatggcgcgattggtcatgccgaatgcgaacgtcgctatcgtcagatcaatgattttgaacgtatgctgagcgaaaccggcaccaccatccgtaaattctatctgcatatcagcaaagatgaacagaaaaaacgtctggaagcgcgtcgcgataccccgaaaaaacgctggaaatttcagccgggtgatctggccgtgcgtgcacagtgggatgattatcgtgccgcctatgatgccctgctgagcgcaaccagcacccgtcatgccccgtggcatgtggttccggcggatgataaactggcccgcaacctgattgtgagcgcgctgctgatcgaagccctggaaggtctggatatgcgctatccggaaccggtggcgggcgttgcgggcaccccgattatctaataa

SEQ ID NO: 20 - DNA, 1092 bp, Pseudomonas fluorescens
atgagcgaagaaagcaccgccctgccgctgccgccggcaccggtgcagaaagcgccggccagcaccagcccggcgagcaccaaagcggcgccgcgtaaagccgcaaccccgcgtccgcgtcgcccgcgtaccaccaaagcggccccggttaaagcggccgaagcggaaattagcgccatcagccagaaaccgatggccctgcaagtggccaatgccccgcgtggcagcaatgaagatagcgttagcgcggccctgccgggtaactatccgtatcgtaatcgtatgcgtcgtgcggaatatgaaaaagccaaaaacgaactgcaaattgaactgctgaaagtgcagagctgggttaaagaaaccggccagcgcatcgtggttctgtttgaaggtcgcgatgcggccggtaaaggcggcaccattaaacgctttatggaacatctgaatccgcgtggcgcgcgtattgtggccctggaaaaaccgagcgaacaggaaaaaggccagtggtattttcagcgttatattcagcatctgccgaccgcgggcgaaatggtgtttttcgatcgtagctggtataaccgcgccggtgtggaacgtgttatggaattttgcagcccgctgcaatatctggaatttatgcgccagaccccggaactggaacgtatgctgtgtaacagcggtattctgatgttcaaattctggttcagcgtgaatcgcgaagaacagctgcgtcgctttatcagccgtcgcgatgatccgctgaaacattggaaactgagcccgattgatatcaaaagcctggataaatgggatgaatataccgcggccaaacaggcgatgtttttccataccgataccgcggatgccccgtggaccgttatcaaaagcgatgataaaaaacgtgcgcgcattaattgcatccgccattttctgcatgaactggattatccgggcaaagatctgaaagtggcccatgcgccggacccgctgctggttggccgtgccagccgcggtctggaagaagatgaacgtacccaggcacaggccgcaaccgatgccggtgcaaccaaactggccctgagcgcataataa